(19) United States
(12) Patent Application Publication
(10) Pub. No.: US 2012/0108965 A1
(43) Pub. Date: May 3, 2012

(54) Facilitating Desired Transducer Manipulation for Medical Diagnostics and Compensating for Undesired Motion

(75) Inventor: Roee Lazebnik, San Jose, CA (US)

(73) Assignee: SIEMENS MEDICAL SOLUTIONS USA, INC., Malvern, PA (US)

(21) Appl. No.: 12/913,588

(22) Filed: Oct. 27, 2010

(51) Int. Cl. A61B 8/00

(52) U.S. Cl. 600/438; 600/459

(57) ABSTRACT

Methods and apparatus are described for facilitating desired transducer manipulation for medical diagnostics and/or compensating for undesired transducer motion. In one embodiment, a transducer is provided with one or more motion sensing elements such as accelerometers, magnetic sensors, etc. During image collection, motion of the transducer is tracked and compared to a desired motion, which may include lack of motion. Feedback may be provided to the operator to facilitate desired manipulation of the transducer. Feedback may be visual feedback, audio feedback or some other form of feedback (for example, tactile feedback). If the operator's technique is deficient, the operator may be prompted to repeat the image collection steps. Various motion templates may be stored according to specific transducer models, examination types, involved anatomy, etc. Motion data may also be used to compensate for undesired motion.

### BACKGROUND

The present invention relates to medical diagnostics, particularly to medical diagnostics using a handheld transducer.

Medical diagnostics often involves the local application of energy to the body through means of a handheld transducer.

In the case of medical imaging, including ultrasound imaging, proper image collection may depend on correct transducer manipulation. This is the case for ultrasound elastography, for example. An elastogram displays tissue elasticity as measured over a tissue volume, for example using brightness or color attributes. Tissue elasticity may be computed by an ultrasound machine based on a series of images of the tissue during palpation of the tissue. Palpation of the tissue may be manual or automated. In the case of manual-compression elastography, palpation is accomplished manually through motion of the transducer. Effective manual-compression elastography may depend on correct transducer motion. Typically, in the case of non-manual compression elastography, palpation is accomplished by the automated application of energy, such as ultrasound energy through the transducer. In non-manual compression elastography, it is desirable that the transducer be held still. In any given instance, the operator may be unclear about how the transducer should be held or manipulated. Furthermore, the operator's technique may be lacking, with the result that image collection and subsequent processing is compromised.

### SUMMARY

Methods and apparatus are described for facilitating desired transducer manipulation for medical diagnostics and/or compensating for undesired transducer motion. In one embodiment, a transducer is provided with one or more motion sensing elements such as accelerometers, magnetic sensors, etc. During image collection, motion of the transducer is tracked and compared to a desired motion, which may include lack of motion. Feedback may be provided to the operator to facilitate desired manipulation of the transducer. Feedback may be visual feedback, audio feedback or some other form of feedback (for example, tactile feedback). If the operator's technique is deficient, the operator may be prompted to repeat the image collection steps. Various motion templates may be stored according to specific transducer models, examination types, involved anatomy, etc. Motion data may also be used to compensate for undesired motion.

### DRAWING FIGURES

FIG. 1 is a diagram of an imaging system;

FIG. 2A is an isometric sketch of a transducer used in the system of FIG. 1;

FIG. 2B is an isometric sketch of another transducer used in the system of FIG. 1;

FIG. 3 is a block diagram of a processor used to process imaging data from the transducer of FIGS. 2A and 2B to generate an image for an operator of the system of FIG. 1;

FIG. 4A is a diagram showing a coordinate system for a one-dimensional array transducer used in the system of FIG. 1;

FIG. 4B is a diagram showing a coordinate system for a two-dimensional array transducer used in the system of FIG. 1;

FIGS. 5A, 5B and 5C show various motions which may be imparted to the transducer by the operator;

FIG. 6A is a diagram illustrating one possible user interface technique for compression elastography;

FIG. 6B and FIG. 6C are diagrams illustrating one possible user interface technique for non-manual compression elastography;

FIG. 7 is a flow diagram of a process that may be used by the processor of FIG. 3.

### DETAILED DESCRIPTION

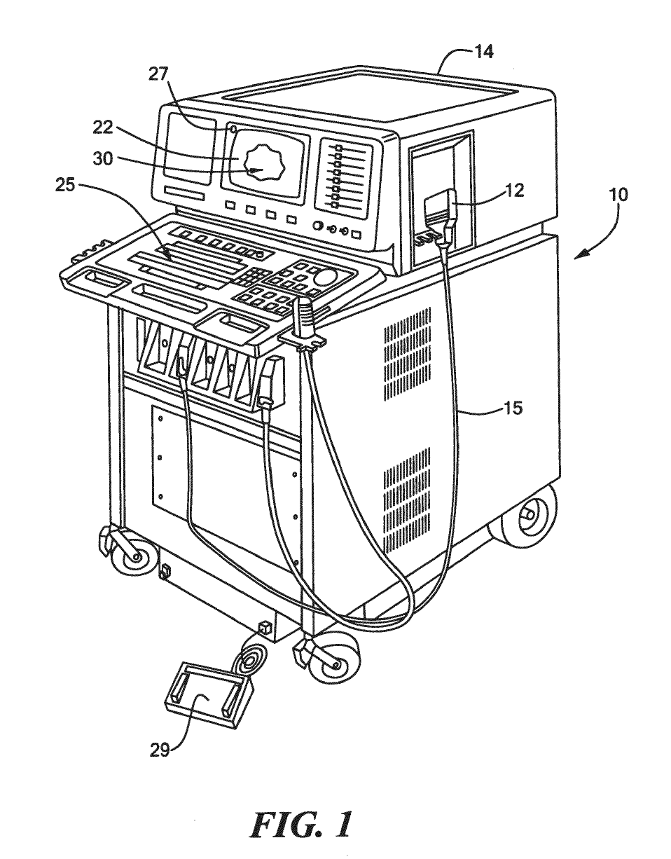

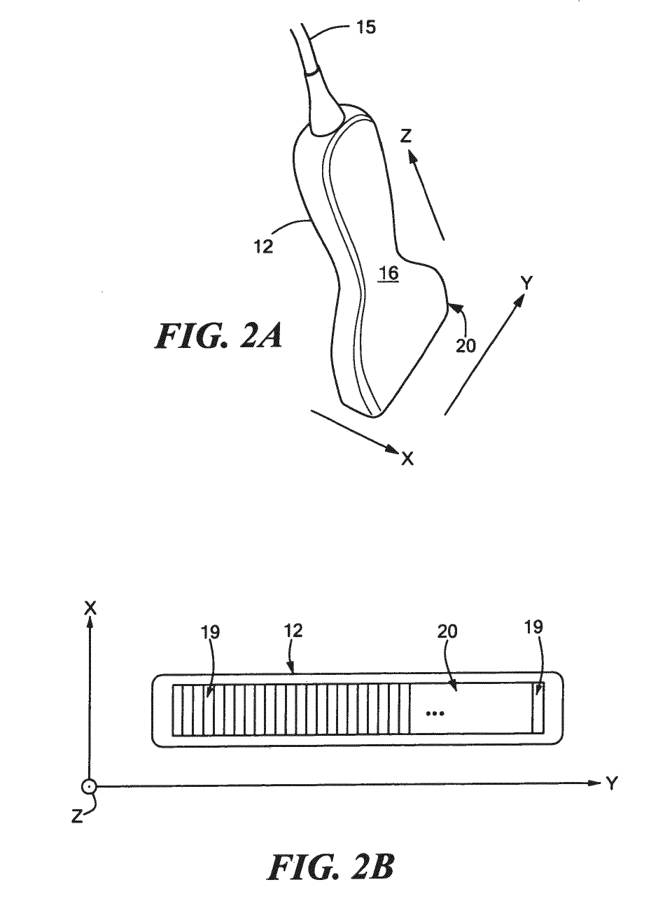

Referring to FIG. 1, a medical diagnostic system 10 is shown, e.g., an ultrasound imaging system. The system 10 includes a positionable, e.g., handheld, image processing ultrasound device in the form of a transducer 12 shown in more detail in FIGS. 2A and 2B, and a multi-use display device, or user interface, herein sometimes collectively referred to as a workstation 14. Control signals may be entered manually by an operator (i.e., sonographer) using a workstation keyboard 25 or by a foot pedal 29. In either case, these controls allow the operator to adjust the operation of the ultrasound machine workstation 14.

The handheld transducer 12 obtains ultrasound data and formats the ultrasound data for transmission to the workstation 14, e.g., via a cable 15. Quality of image collection may be affected by motion or patterns of motion imparted to the transducer 12 by the operator of the system 10. The pattern of motion may include an up-down/down-up motion of the transducer to achieve tissue palpation. The detection of operator-imparted transducer motions may be performed by hardware and/or software to detect patterns in real time. When motion is detected, various attributes or motion characteristics such as the amplitude of the motion, the timing or frequency of the motion, etc., may be used to discriminate between desired and undesired motion.

In one exemplary embodiment shown in FIG. 2A, the transducer 12 includes a housing 16 adapted to be easily handheld, such as for example, being less than 8 inches in any dimension and/or having an ergonomic shape for holding in a operator's hand. The housing 16 may comprise plastic, rubber, metal, other materials now known or later developed, or combinations thereof. The housing 16 may have a generally rounded or curved-circumference handle serving as a grip for the operator's hand. The housing 16 of the transducer 12, and more particularly a frontal portion 20 thereof, may be rectangular shaped, having its longer dimension along, e.g., a Y, or azimuthal axis, and its shorter dimension along, e.g., the X, or elevation, axis, as indicated. An axial Z axis is thus along the length of the housing (i.e., an axis perpendicular to both the X and Y axes) to provide a conventional Cartesian coordinate system for the transducer 12.

In another exemplary embodiment shown in FIG. 2B, the transducer 12 is provided with accelerometers such as micromachined accelerometers 13, and 17, one disposed within the housing 16 of the transducer 12 along the Y axis, and other disposed, for example, along Z axis, as shown. An additional accelerometer (not shown) may be disposed along the X axis. The accelerometers may be discrete devices such as model series ADXL manufactured by Analog Devices, Norwood, Mass. Alternatively, the accelerometers may be integrated as part of a single integrated circuit. Accelerometer data from the accelerometers may be transmitted to processing and control circuitry through the cable 15, for example.

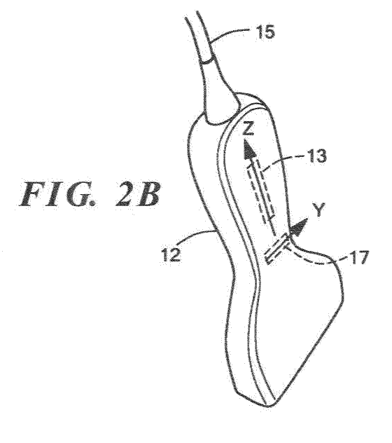

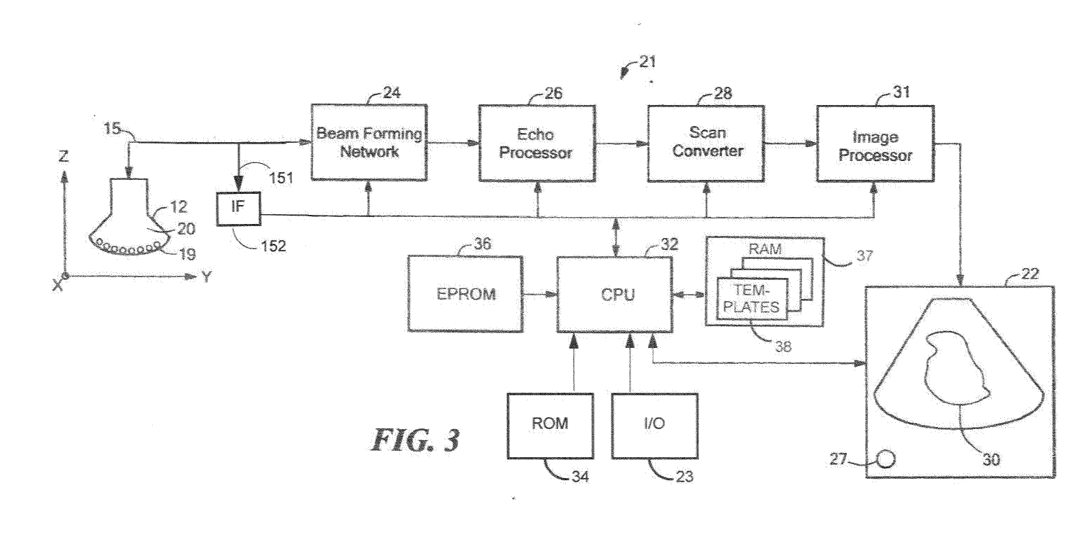

Referring to FIG. 3, the handheld transducer 12 may include conventional ultrasound circuitry in a frontal portion 20 thereof such as an array of ultrasonic elements 19 which transmit and receive ultrasonic energy for imaging a portion of the body of a patient, not shown. The transducers of FIG. 2A and FIG. 2B may be one-dimensional array transducers or 2D array transducers. Information derived from the elements 19 in the frontal portion 20 may be processed by an ultrasound processor 21 and output as an image on a display 22 of the workstation 14 (FIG. 1) serially through: a beamforming network 24, an echo processor 26, a scan converter 28, and an image processor 31. The beamforming network 24, echo processor 26, scan converter 28, image processor 31 and display 22 may be controlled by a central processing unit (CPU) 32 coupled to a random access memory RAM 37. The CPU 32 may operate in accordance with program instructions stored in a ROM 34, or in RAM 37, or in flash memory not shown, or on a hard drive device, not shown. A memory 36 such as an erasable programmable semiconductor memory, a read only memory (ROM), etc., may be provided for storing a computer- or microprocessor-executable program, for operating the CPU 32 as described herein. Further, the RAM 37 may store (e.g., after being read from the hard drive, not shown) various templates 38 describing desired transducer motion under different conditions such as specific transducer models, examination types, involved anatomy, etc. Detected motion imparted by the operator of the transducer 12 may be compared to a selected template to discriminate between desired and undesired motion.

The operator may alter the selected template using setup screen touch control signals. More particularly, since desired elastographic technique can vary between organs, different templates may be provided to allow feedback to the operator to be customized on a per-clinical-application basis or use-condition basis as specified, for example, by an operator-selected preset or the like.

In one exemplary embodiment, the ultrasound processor 21 scan converts data associated with a radial scan pattern to generate ultrasound image data in a video format (e.g. Cartesian coordinate format). The processor 21 is coupled to the array of transmitting/receiving elements 19, e.g., an array of piezoelectric crystals that deliver ultrasonic energy into a patient and receive ultrasonic echoes from the patient. These elements 19 are arranged to form a one-dimensional, 1.5D, two-dimensional or single element transducer. Any of a phased array, linear array, curved array or other arrays may be used. An acoustic window, not shown, may be disposed in the frontal portion 20 on the housing 16 (FIG. 2A, FIG. 2B) of the transducer 12.

In operation, electrical signals representative of echoes produced by the transducer 12 are delivered to the beamforming network 24 where they are selectively combined to produce an indication of the echo intensity along a particular direction or beam in the patient. The data produced by the beamforming network 24 may be fed to the echo processor 26 which calculates echo intensity at each position along a beam and may calculate a Doppler shift of the echoes received along a particular beam. Data from the echo processor 28 may be fed to a scan converter 28 that converts the data into a form that can be readily displayed on a video monitor 22. Other I/O circuitry 23 may include keyboards, mice or other cursoring devices, touchscreens, touchpads, or any of various other types of human interface devices including wireless audio or video devices.

The data produced by the scan converter 28 may be stored in the RAM 37 where additional processing, such as adding color, may be performed prior to displaying the images on a video monitor. In the case of elastography, data of multiple scans may be used in combination to compute elastography data and to produce an elastogram for display. Controlling the operation of the above-referenced parts are one or more central processing units, collectively indicated by the CPU 32. The central processing units also receive control signals from the operator.

Motion detection signals may be input from the transducer 12 via cable 15, a signal path 151, and an interface 152. Alternatively, motion detection signals may be input from the transducer wirelessly.

The CPU 32, with access to the image data stored in RAM 37 and the templates stored in memory 36, processes motion detection signals describing motion imparted by the operator to the transducer, such processing being used to provide workstation control signals. Recognition by the processor 21 of the motion signals as matching a selected template may result, for example, in the CPU 32 sending a signal to a light and/or chime 27 mounted on the workstation 14, or changing some on-screen indicator. Activation of the light and/or chime or on screen indicator 27 provides a visual and/or audible indication to the operator that image capture has been successfully completed. Similarly, recognition of the motion of the transducer by the processor 21 as not matching a selected template may result, for example, in the CPU 32 sending a signal to a light and/or buzzer 27 mounted on the workstation 14, or changing some on-screen indicator. Activation of the light and/or buzzer or on screen indicator 27 provides a visual and/or audible indication to the operator that image capture has not been successfully completed, or that the captured image data may be lacking in quality.

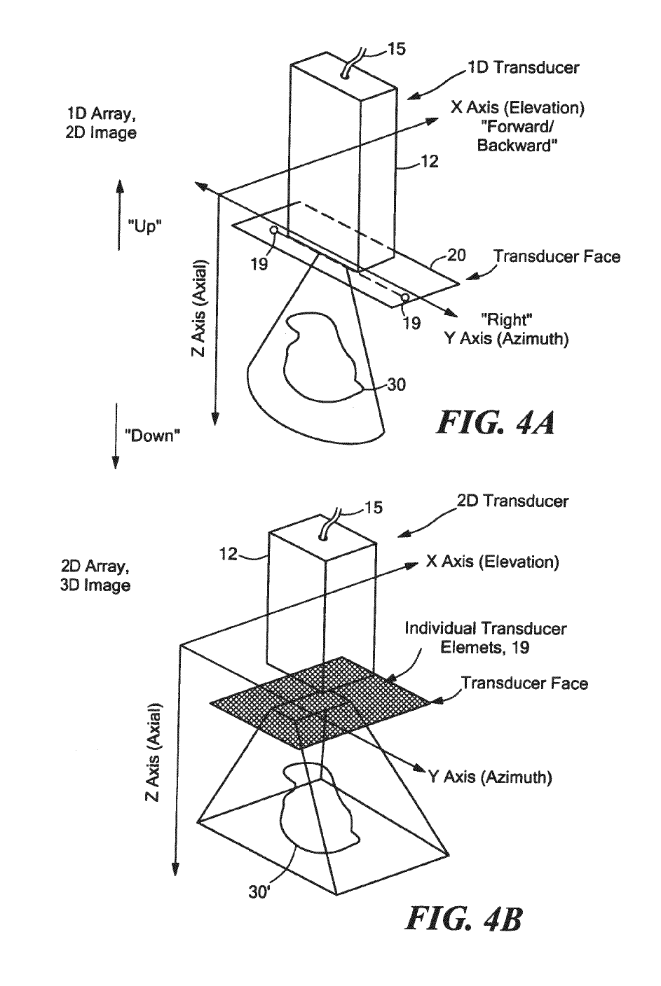

FIG. 4A shows the region of a scan of an image 30, e.g., a sonogram, produced by placing the transducer 12 at one fixed position on the patient's body, not shown. The transducer 12 shown in FIG. 4A has a one-dimensional array of the transmit/receive elements 19. The image 30 is the Y-Z plane of the transducer's coordinate system.

FIG. 4B shows the region of a scan of an image 30 produced by a transducer 12 having a two dimensional array of elements 19. Note that image 30′ produced by this two dimensional array transducer is a three-dimensional image 30′.

As described above, the transducer 12 may be electrically coupled to the workstation 14 (FIG. 1) by a cable 15. In other embodiments, the transducer 12 may be wirelessly coupled to the workstation 14 as described in U.S. Pat. No. 6,780,154 issued Aug. 24, 2004, inventors Hunt et al., assigned to the same assignee as the present invention, the entire subject matter thereof being incorporated herein by reference.

Furthermore, the I/O circuitry 23 may include wireless human interface devices such as, for example, a wireless earpiece wirelessly connected via a wireless personal area network technology such as BlueTooth or the like. At the start of an elastographic examination, the earpiece may be used to remind an operator of the correct technique for the selected examination. The earpiece may also transmit sound effects prompting the operator to execute the correct technique and providing feedback to the operator. In the case of compression elastography, for example, the earpiece may provide an audible beat having a frequency equal to a desired frequency of motion of the transducer, for example 20 beats per minute. In the case of non-manual compression elastography, the earpiece may provide a noise signal proportional to undesired motion of the transducer such that the operator strives to hold the transducer sufficiently still to achieve quiet. As needed, the operator may be prompted using verbal prompts. Since these prompts are audible only to the operator, there is no embarrassment on the part of the operator or uneasiness on the part of the patient.

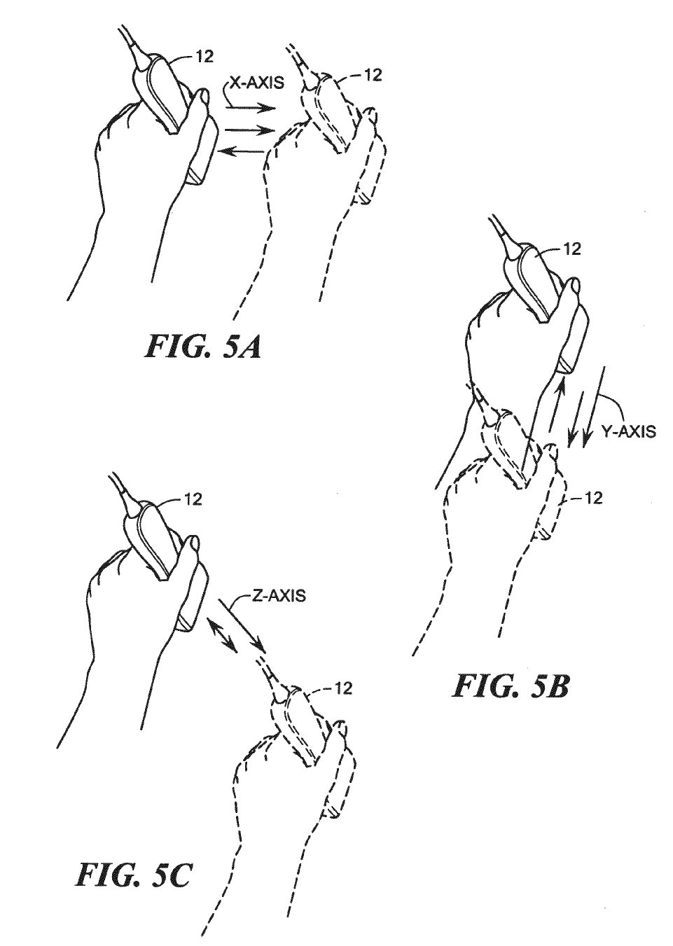

FIG. 5A shows of a motion of the transducer 12 by the operator along the X-axis (i.e., a forward/backward motion); FIG. 5B shows of a motion of the transducer 12 by the operator along the Y-axis (i.e., a right (R)/left (L) motion; and, FIG. 5C shows of a motion of the transducer 12 by the operator along the Z-axis (i.e., an up (U)/down (D) motion.

As noted above, the processor 21 (FIG. 3) detects patterns of these X, Y and/or Z operator imparted motions to provide controls to the workstation 14. Software and/or hardware may be used to detect patterns of transducer motion, and hardware and/or software may be used to map those patterns to the activation of system controls. When motion is detected, the extent and timing of the motion may be used to discriminate between desirable motion and undesirable motion during scanning.

Motion detection may be performed in any of a variety of ways. In one embodiment, accelerometers are provided within the housing 16 of the transducer 12. In another embodiment, magnetic sensors are provided within the housing 16 of the transducer 12. In another embodiment, one or more rate gyros for sensing twisting or rolling motion may be disposed within the housing 16 of the transducer 12, such as a model series ADSRS manufactured by Analog Devices, or other motion-sensing devices may be disposed within the housing 16 of the transducer 12. A video monitoring camera may be used to detect motion. Another technique may include mounting light emitting diodes to the transducer body and having light detecting sensors fixed to the workstation or to the examination room, apart from the transducer. One such system is manufactured by Northern Digital (NDI), International Headquarters 103 Randall Drive Waterloo, Ontario Canada N2V 1C5. Whatever the mechanism of sensing motion, signals describing motion of the transducer 12 may be coupled from the transducer 12 to the workstation 14, either through cable 15 or wirelessly.

Moreover, the detection of motion may be performed using image data itself. For example, the detection of transducer motion may be done using decimated image data; using Doppler Tissue Imaging, in which dedicated hardware or software averages the computed Doppler velocity and/or Doppler energy signals from a sample set of echo information at a predetermined set of image locations; or in a like manner but with the predetermined set of image locations confined to the near-field of the image.

Another technique that may be used to detect transducer motion is described in U.S. Pat. No. 6,162,174 entitled “Method for compensating for object motion in ultrasound images”, issued Dec. 19, 2000, inventor Friemel, assigned to the same assignee as the present invention, the entire subject matter thereof being incorporated herein by reference. While there transducer motion is detected to remove image flicker, the method includes determining transducer motion. As noted above, when motion is detected, the extent and timing of the motion are be used to discriminate between desired motion and undesired motion during scanning.

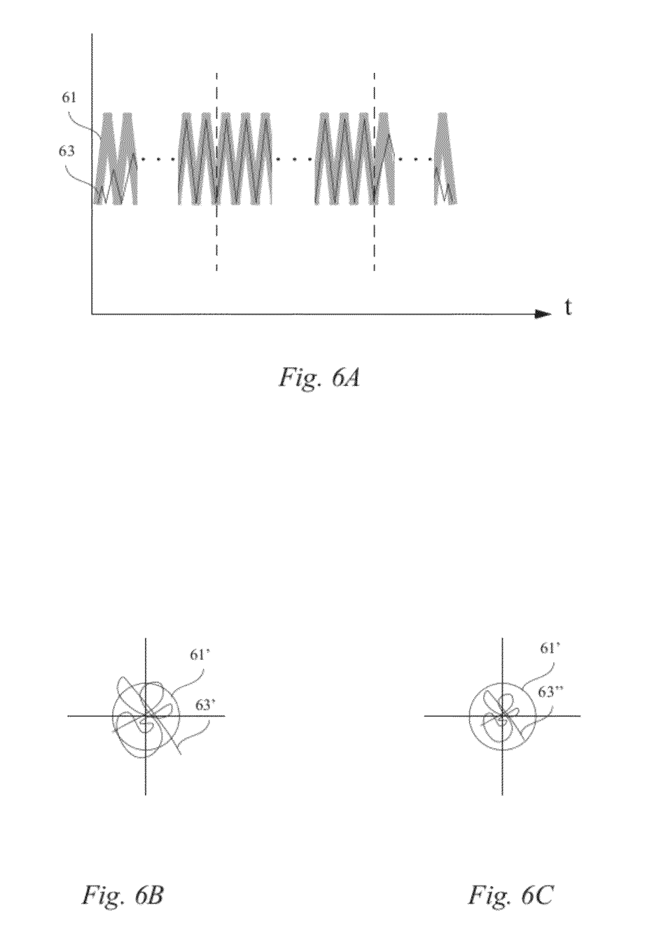

FIG. 6A illustrates one possible user interface technique for manual-compression elastography. A graphical representation may be provided of a desired motion template. In this example, the desired motion is an oscillatory motion having a target amplitude and a target frequency. A band 61, illustrated in gray, indicates a desired region within which a trace 63 representing motion of the transducer by the operator is desired to appear. At the outset, the motion of the transducer may not fall within the desired parameters. A red shading may be displayed on the screen during this time and image capture may be postponed. Feedback to the operator enables the operator to bring the motion of the transducer within the desired parameters. At this time, a green shading may be displayed on the screen and image capture may proceed. At the end of image capture, as determined either by the operator or by the system, either the system produces an indication that image capture is complete, causing the operator to quit moving the transducer in the same manner as before, or the operator recognizes that image capture is complete and quits moving the transducer in the same manner as before. At this time, a red shading may again be displayed.

FIGS. 6B and 6C illustrate one possible user interface technique for non-manual compression elastography. In this example a cross-hair display is provided and motion of the transducer, which is undesirable, is displayed in relation to the cross hair. In the illustrated example, the cross-hair display is two dimensional, but in practice, it may be three-dimensional if desired. A circle 61′ indicates bounds within which slight motion may be acceptable. In FIG. 6B, these bounds are exceeded by a trace 63′ representing motion of the transducer, and a red shading may be displayed on the screen. Feedback to the operator enables the operator to bring the motion (stillness) of the transducer within the desired parameters. At this time (FIG. 6C), the trace 63″ is within the bounds of the circle 61′; a green shading may be displayed on the screen and image capture may proceed.

Any of a great variety of operator techniques may be used to facilitate desired transducer motion or lack thereof. Other examples include providing a numerical score describing technique quality, displaying a graphical display of motion data, providing a “traffic light” type display, audible prompts, etc.

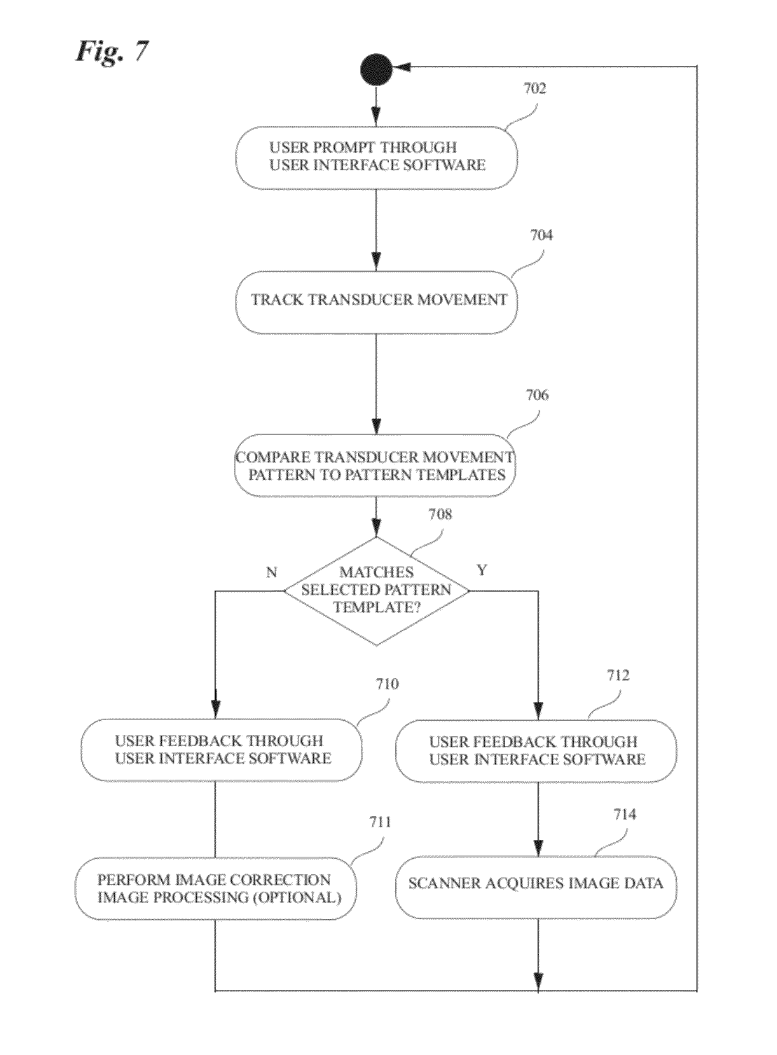

Referring now to FIG. 7, a flow diagram of one method used herein to generate workstation control signals from operator imparted motions to the transducer is shown.

A operator prompt may be optionally provided through user interface software (Step 702). The processor 21 (FIG. 3) tracks transducer movement (Step 704). The movement is compared to a selected pattern template (Step 706).

Based on the comparison results (Step 708), corresponding control signals are provided to the workstation 14. If the movement matches the template, operator feedback may be provided through the user interface software (Step 712), and the scanner may proceed to acquire image data (Step 714). If the movement does not match the template, operator feedback may be provided through the user interface software (Step 710), with or without the scanner acquiring image data.

Optionally, as shown in Step 711, if undesired transducer motion has occurred during acquisition of image data, stored motion data describing the undesired motion of the transducer may be used to perform image correction processing. Referring again to FIG. 3, the CPU 32, having detected undesired motion, may issue commands to the image processor 31 to cause the image processor to perform image correction processing. Such image correction may use techniques described, for example in U.S. Pat. No. 6,641,536 of the present assignee, incorporated herein by reference, or other image correction processing techniques.

A number of embodiments of the invention have been described. Nevertheless, it will be understood that various modifications may be made without departing from the spirit and scope of the invention.

What is claimed is:

1. A user interface method for a medical diagnostic machine comprising a handheld transducer, the method comprising:
detecting motion or lack thereof of the handheld transducer;
comparing said motion or lack thereof of the handheld transducer to a desired motion characteristic; and
providing feedback to an operator according to results of said comparing;
wherein the desired motion characteristic is one of:
a manual compression motion used in manual-compression elastography; and
a lack of motion used in non-manual compression elastography.

2. The method of claim 1, comprising:
performing acquisition of elastography data; and
using data describing motion of the handheld transducer to correct the elastography data for motion of the handheld transducer.

3. The method of claim 1, comprising:
inputting an operator selection corresponding to a use condition of the medical diagnostic machine; and
customizing said feedback to the operator according to the use condition.

4. The method of claim 3, wherein the use condition includes at least identification of a body organ targeted during operation of the medical diagnostic machine.

5. The method of claim 1, wherein the feedback is automated audio or video feedback.

6. The method of claim 1, comprising providing an automated audio or video prompt indicative of a desired manipulation of the handheld transducer.

7. The method of claim 1, comprising providing an automated audio indication of a desired manipulation of the handheld transducer and sending the automated audio indication to a wireless earpiece of an operator.

8. The method of claim 1, comprising automatically tracking motion of the handheld transducer.

9. The method of claim 1, wherein the medical diagnostic machine is a medical imaging machine.

10. The method of claim 9, wherein the medical imaging machine is an ultrasound machine.

11. A processing method carried out by a medical diagnostic machine comprising processing circuitry, memory circuitry, and input/output circuitry, the method comprising:
the processing circuitry detecting motion or lack thereof of a handheld transducer in accordance with signals input from the handheld transducer;
the processing circuitry comparing said motion or lack thereof of the handheld transducer to a desired motion characteristic; and
the processing circuitry controlling the input/output circuitry to provide feedback to an operator according to results of said comparing;
wherein the desired motion characteristic is one of:
a manual compression motion used in manual-compression elastography; and
a lack of motion used in non-manual compression elastography.

12. The method of claim 11, comprising:
the processing circuitry performing acquisition of elastography data; and
the processing circuitry using data describing motion of the handheld transducer to correct the elastography data for motion of the handheld transducer.

13. The method of claim 11, comprising:
the I/O circuitry inputting an operator selection corresponding to a use condition of the medical diagnostic machine; and
the processing circuitry customizing said feedback to the operator according to the use condition.

14. The method of claim 13, wherein the use condition includes at least identification of a body organ targeted during operation of the medical diagnostic machine.

15. The method of claim 11, comprising outputting from the processing circuitry to the input/output circuitry data representing audio or video operator feedback resulting from comparison of actual manipulation of the handheld transducer with a desired manipulation of the handheld transducer.

16. The method of claim 11, wherein a plurality of motion templates are stored in the memory circuitry, comprising selecting a motion template in accordance with at least one of: a transducer model; a type of examination; and an involved anatomy.

17. The method of claim 11, comprising the processing circuitry making a prediction of results quality based on said comparing, and outputting result data to external storage only if said quality is predicted to be acceptable.

18. The method of claim 11, comprising the processing circuitry making a prediction of results quality based on said comparing, and outputting result data to external storage together with an indication of said prediction.

19. A medical diagnostic machine for use with a handheld motion-sensing transducer, comprising:
input circuitry for inputting motion signals from the handheld motion-sensing transducer;
processing circuitry coupled to the input circuitry for comparing motion or lack thereof described by the motion signals to a desired motion characteristic; and
output circuitry coupled to the processing circuitry for providing feedback to an operator according to results of said comparing;
wherein the desired motion characteristic is one of:
a manual compression motion used in manual-compression elastography; and
a lack of motion used in non-manual compression elastography.

20. The apparatus of claim 19, wherein the processing circuitry is configured to:
performing acquisition of elastography data; and
using data describing motion of the handheld transducer to correct the elastography data for motion of the handheld transducer.

21. The apparatus of claim 19, wherein:
the input circuitry is configured to input an operator selection corresponding to a use condition of the medical diagnostic machine; and
the processing circuitry is configured to customize said feedback to the operator according to the use condition.

22. The apparatus of claim 21, wherein the use condition includes at least identification of a body organ targeted during operation of the medical diagnostic machine.

23. The apparatus of claim 19, wherein the processing circuitry and memory circuitry are configured to provide an automated audio or video prompt indicative of a desired manipulation of the handheld transducer.

24. The apparatus of claim 19, wherein the memory circuitry is configured to store a plurality of motion templates, and the processing circuitry is configured to select a motion template in accordance with at least one of: a transducer model; a type of examination; and an involved anatomy.

25. The apparatus of claim 19, wherein the medical diagnostic machine is a medical imaging machine.

26. The apparatus of claim 25, wherein the medical imaging machine is an ultrasound machine.

27. A computer-readable medium for providing user interface for a medical diagnostic machine comprising a handheld transducer, comprising instructions for:
detecting motion or lack thereof of the handheld transducer;
comparing said motion or lack thereof of the handheld transducer to a desired motion characteristic; and
providing feedback to an operator according to results of said comparing;
wherein the desired motion characteristic is one of:
a manual compression motion used in manual-compression elastography; and
a lack of motion used in non-manual compression elastography.

28. The apparatus of claim 27, comprising instruction for:
performing acquisition of elastography data; and
using data describing motion of the handheld transducer to correct the elastography data for motion of the handheld transducer.

29. The apparatus of claim 27, comprising instructions for:
inputting an operator selection corresponding to a use condition of the medical diagnostic machine; and
customizing said feedback to the operator according to the use condition.

30. The apparatus of claim 29, wherein the use condition includes at least identification of a body organ targeted during operation of the medical diagnostic machine.

31. The apparatus of claim 27, comprising instructions for providing an automated audio or video prompt indicative of a desired manipulation of the handheld transducer.

32. The apparatus of claim 27, comprising instructions for providing an automated audio indication of a desired manipulation of the handheld transducer and sending the automated audio indication to a wireless earpiece of an operator.

33. The apparatus of claim 27, comprising instructions for automatically tracking motion of the handheld transducer.

34. The apparatus of claim 27, wherein the medical diagnostic machine is a medical imaging machine.

35. The apparatus of claim 34, wherein the medical imaging machine is an ultrasound machine.
